SEQUENCE LISTING (3 sequences)

SEQ ID NO: 1 - DNA, 20 bp, synthetic construct
  source 1..20: mol_type = other DNA
agagtttgatcctggctcag

SEQ ID NO: 2 - DNA, 20 bp, synthetic construct
  source 1..20: mol_type = other DNA
tacgacttaaccccaatcgc

SEQ ID NO: 3 - DNA, 1422 bp, Shewanella decolorationis
  source 1..1422: mol_type = genomic DNA
ggcgcgcggctacacatgcagtcgagcggcagcacaagtgagtttactcatgaggtggcgagcggcggacgggtgagtaatgcctagggatctgcccagtcgagggggataacagttggaaacgactgctaataccgcatacgccctacgggggaaaggaggggaccttttggccttccgcgattggatgaacctaggtgggattagctagttggtgaggtaatggctcaccaaggcgacgatccctagctgttctgagaggatgatcagccacactgggactgagacacggcccagactcctacgggaggcagcagtggggaatattgcacaatgggggaaaccctgatgcagccatgccgcgtgtgtgaagaaggccttcgggttgtaaagcactttcagtagggaggaaaggttgtaagttaataccttgcagctgtgacgttacctacagaagaaggaccggctaactccgtgccagcagccgcggtaatacggagggtccaagcgttaatcggaattactgggcgtaaagcgtgcgcaggcggtttgttaagcgagatgtgaaagccccgggctcaacctgggaattgcatttcgaactggcaaactagagtcttgtagaggggggtagaattccaggtgtagcggtgaaatgcgtagagatctggaggaataccggtggcgaaggcggccccctggacaaagactgacgctcaggcacgaaagcgtggggagcaaacaggattagataccctggtagtccacgccgtaaacgatgtctactcggagtttggtgtcttgaacactgggctctcaagctaacgcattaagtagaccgcctggggagtacggccgcaaggttaaaactcaaatgaattgacgggggcccgcacaagcggtggagcatgtggtttaattcgatgcaacgcgaagaaccttacctactcttgacatccagagaactttccagagatggattggtgccttcgggaactctgagacaggtgctgcatggctgtcgtcagctcgtgttgtgaaatgttgggttaagtcccgcaacgagcgcaacccctatccttatttgccagcgcgtaatggcgggaactctagggagactgccggtgataaaccggaggaaggtggggacgacgtcaagtcatcatggcccttacgagtagggctacacacgtgctacaatggcgagtacagagggttgcaaagccgcgaggtggagctaatctcacaaagctcgtcgtagtccggattggagtctgcaactcgactccatgaagtcggaatcgctagtaatcgtgaatcagaatgtcacggtgaatacgttcccgggccttgtacacaccgcccgtcacaccatgggagtgggctgcaaaagaagtgggtagcttaacctcgggagggcgc

(19) United States
(12) Patent Application Publication
(10) Pub. No.: US 2024/0200020 A1
(43) Pub. Date: Jun. 20, 2024

(54) SHEWANELLA DECOLORATIONIS PRODUCING TETRODOTOXIN AND APPLICATION THEREOF

(71) Applicant: Yellow Sea Fisheries Research Institute, Chinese Academy of Fishery Sciences, Qingdao (CN)

(72) Inventors: Changwei Shao, Qingdao (CN); Sheng Du, Qingdao (CN); Qian Wang, Qingdao (CN); Hongyan Wang, Qingdao (CN); Kaiqiang Liu, Qingdao (CN); Yuyan Liu, Qingdao (CN)

(21) Appl. No.: 18/518,137

(22) Filed: Nov. 22, 2023

(30) Foreign Application Priority Data
Dec. 20, 2022 (CN) 202211638761.0

(51) Int. Cl. C12N 1/20

(52) U.S. Cl. CPC C12N 1/205; C12N 2500/74

(57) ABSTRACT

Disclosed are a Shewanella decolorationis producing tetrodotoxin and an application thereof, falling in the field of development and utilization of medicinal microorganisms. A strain, Shewanella decolorationis S3-4, is deposited in the China General Microbiological Culture Collection Center (CGMCC) on Mar. 28, 2022, with a deposit number of CGMCC No. 24602. The strain can secrete a same substance as tetrodotoxin from Tetraodontidae.

### REFERENCE TO SEQUENCE LISTING

The replacement sequence listing is submitted as a XML file filed electronically via the Patent Center, with a file name of “Replacement Sequence Listing.XML”, a creation date of Dec. 22, 2023, and a size of 5,355 bytes. The replacement sequence listing is a part of the specification and is incorporated in its entirety by reference herein.

### CROSS-REFERENCE TO RELATED APPLICATIONS

This application claims priority of Chinese Patent Application No. 202211638761.0, filed on Dec. 20, 2022, the entire contents of which are incorporated herein by reference.

### TECHNICAL FIELD

The present disclosure relates to the field of development and utilization of medicinal microorganisms, in particular to a Shewanella decolorationis producing tetrodotoxin and an application thereof.

### BACKGROUND

Tetradotoxin (TTX) is a small molecular weight non-protein neurotoxin with short incubation period and high mortality rate. After being absorbed, tetrodotoxin quickly acts on the peripheral nerves and the central nervous system, specifically blocking voltage-gated sodium channels on cell membranes of nerve cells, disordering nerve conduction and paralyzing sensory nerves and motor nerves, and in severe cases, brainstem paralysis leads to respiratory and circulatory failure. Tetrodotoxin is contained in different tissues such as epidermis, viscus, blood, testis, ovary, liver, spleen and eyeballs of Tetraodontidae.

Because of the property of tetrodotoxin specifically blocking the voltage-gated sodium channels, tetrodotoxin can be potentially used as medicines for pain, anesthesia, detoxification, beauty and so on. TTX is often used for pain treatment, blood pressure reduction, anti-arrhythmia, local anesthesia, detoxification and tumor suppression in clinic. However, the synthetic tetrodotoxin is costly. At present, most of raw materials for extracting tetrodotoxin come from internal organs of wild Tetraodontidae, but it is difficult to be extracted due to the limited number of wild Tetraodontidae, and the killing of the wild Tetraodontidae will destroy the natural resources of Tetraodontidae.

With the in-depth study of tetrodotoxin, the “exogenous origin theory” of tetrodotoxin has been continuously confirmed. Most researchers believe that TTX in the Tetraodontidae results from the joint action of food chain and microorganisms in Tetraodontidae body. In China, it has been reported that researchers have isolated bacteria capable of producing tetrodotoxin from the liver and gonads of Takifugu rubripes and Takifugu obscurus, clearly identifying that the tetrodotoxin produced by the aeromonas capable of producing tetrodotoxin isolated from the tissues of Takifugu rubripes and Takifugu obscurus is the same as the tetrodotoxin extracted from Tetraodontidae.

Although there have been studies on the microorganisms producing tetrodotoxin in China, the alternative microorganisms and cultivation method thereof are extremely limited for large-scale industrial production, which seriously limits the development of industrial production of tetrodotoxin.

### SUMMARY

Aiming at the limitation of industrial production of tetrodotoxin, the present disclosure provides a Shewanella decolorationis S3-4 capable of producing tetrodotoxin, isolated from the liver, ovary and intestines of a wild Takifugu ocellatus. The Shewanella decolorationis S3-4 can secrete tetrodotoxin.

The present disclosure is realized by the following technical solutions.

A Shewanella decolorationis producing tetrodotoxin, a strain of S3-4, is classified as Shewanella decolorationis and deposited in the China General Microbiological Culture Collection Center (CGMCC) on Mar. 28, 2022 with a deposit number of CGMCC No. 24602.

The present disclosure also provides a method for producing tetrodotoxin by utilizing Shewanella decolorationis, including the following steps: inoculating Shewanella decolorationis S3-4 into an LB liquid culture medium; and culturing a same at 28° C. at 200 rpm for 2-3 days.

Further, the LB liquid culture medium includes the following components: tryptone with a final concentration of 10.0 g/L, yeast extract powder with a final concentration of 5.0 g/L, and sodium chloride with a final concentration of 10.0 g/L; and the strain of Shewanella decolorationis S3-4 obtained by fermentation culture in the LB liquid culture medium is crushed, isolated and purified to obtain tetrodotoxin.

Compared with the related art, the present disclosure has the following beneficial effects.

The bacteria, Shewanella decolorationis S3-4, provided by the present disclosure can secrete the same substance as tetrodotoxin from Tetraodontidae.

### BRIEF DESCRIPTION OF THE DRAWINGS

FIG. 1 is a bacterial identification diagram of a full-length 16SrDNA of Shewanella decolorationis S3-4 bacterium.

FIG. 2 is an ion chromatography-mass spectrometry diagram of a tetrodotoxin standard substance (0.5 ng/ml).

FIG. 3 is a graph of detection standard.

FIG. 4 is a liquid mass spectrum diagram of a bacterial source sample of Shewanella decolorationis S3-4.

### DETAILED DESCRIPTION

Technical solutions of the present disclosure will be further explained by the following examples, but the protection scope of the present disclosure is not limited by the examples in any form.

### Example 1

Culture media used in the example included: 2216E solid culture media, TCBS solid culture media, LB solid culture media and LB liquid culture media, which were all commercial culture media and purchased from Qingdao Hi-tech Industrial Park Hope Bio-technology Co., Ltd.

Takifugu ocellatus is a small warm-water carnivorous bottom fish, which mainly inhabites nearshore waters and sometimes enters freshwater rivers and brackish-freshwater estuaries. The Takifugu ocellatus has an air bag, which could make its abdomen swell for self-defense when it is attacked by an enemy. The Takifugu ocellatus preys on small shellfish, crablat, amphipods, small crayfish and algae debris. When entering fresh water, the Takifugu ocellatus preys on shrimps, crabs, mussels, fry, aquatic insect larvae, angular and copepods, and occasionally aquatic vascular plants and filamentous algae. During the spawning period from April to June, fish schools migrate to the middle and lower reaches of rivers to spawn, laying demersal and viscid eggs, which belongs to one-time spawning type. The Takifugu ocellatus is hypertoxic in ovary and liver, non-toxic in testis, highly toxic in skins and intestines, and weakly toxic in muscle. In the present disclosure, the ovary, liver and intestines of the Takifugu ocellatus with strong toxicity were selected for research.
(1) A wild Takifugu ocellatus was selected and collected by the inventor from the estuary of Yifeng River in Shantou City, Guangdong Province. Ovarian tissues, liver and intestinal tissues of Takifugu ocellatus containing tetrodotoxin (TTX) were taken.
(2) Tissue homogenate was performed, and diluted solution was prepared with PBS at volume-mass ratios of 1:10, 1:100 and 1:1000. 100 μl of the diluted solution was taken and coated on the 2216E solid culture medium, TCBS solid culture medium and LB solid culture medium.
(3) After culturing at 28° C. for 48 hours, characteristic colonies on a plate were picked out and streaked on another 2216E solid culture medium, TCBS solid culture medium and LB solid culture medium.
(4) After culturing at 28ºC for 24 hours, an isolated single colony was streaked again for isolated culture.
(5) Streaking culture was repeated until the single colony was isolated.

The tissues in step (1) were the liver, ovary and intestines of the Takifugu ocellatus.

The 2216E solid culture medium included the following components: 5.0 g/L of peptone, 1.0 g/L of yeast powder, 0.1 g/L of ferric citrate, 19.45 g/L of sodium chloride, 5.98 g/L of magnesium chloride, 3.24 g/L of sodium sulfate, 1.8 g/L of calcium chloride, 0.55 g/L of potassium chloride, 0.16 g/L of sodium carbonate, 0.08 g/L of potassium bromide, 0.034 g/L of strontium chloride, 0.022 g/L of boric acid, 0.004 g/L of sodium silicate, 0.0024 g/L of sodium fluoride, 0.0016 g/L of ammonium nitrate, 0.008 g/L of disodium hydrogen phosphate and 15.0 g/L of agar.

The TCBS solid culture medium included the following components: 5.0 g/L of yeast extract powder, 10.0 g/L of peptone, 10.0 g/L of sodium thiosulfate, 10.0 g/L of sodium citrate, 5.0 g/L of Ox gallbladder powder, 3.0 g/L of natrii tauroglycocholas, 20.0 g/L of sucrose, 10.0 g/L of sodium chloride and 1.0 g/L of iron citrate, 0.04 g/L of bromothymol blue, 0.04 g/L of thymol blue and 15.0 g/L of agar.

The LB liquid culture medium included the following components: 10.0 g/L of tryptone, 5.0 g/L of yeast extract powder, 10.0 g/L of sodium chloride and 15.0 g/L of agar.

A strain was inoculated into the LB liquid culture medium, and cultured at 28° C. and 200 rpm for 2-3 days. A fermentation broth was detected by liquid chromatography-mass spectrometry, and it was found that a strain S3-4 could produce tetrodotoxin. The strain had the following morphological, physiological and biochemical characteristics.

### (1) Morphological Characteristics of Bacteria

According to the conventional physiological and biochemical identification methods of bacteria, cells of Shewanella decolorationis were Gram-negative bacteria, with a rod-shape and a size of about (0.6-1.0)×(1.0-6.7) μm, having cilia all over the body and a single polar flagellum.

### (2) Main Physiological and Biochemical Characteristics

The strain had ability of facultative anaerobic growth, reducing trivalent iron, liquefying gelatin, and producing H2S. The oxidase and contact enzyme were positive, and could grow with sodium lactate, D-galactose and succinic acid as the only carbon source, but could not grow with ammonium sulfate and ammonium nitrate as the only nitrogen source.

### (3) A Method for Identifying Bacteria by a 16SrDNA Full-Length Sequence:

the DNA of the genome of the strain was obtained by extraction and isolation, 16SrDNA was amplified by PCR, and an upstream primer: 5′-agagtttgatcctggctcag-3′, a downstream primer: 5′-tacgacttaaccccaatccgc-3′, and 50 μl of a PCR reaction system were used. The PCR reaction system included: 25 μl of 2×Rapid Taq Master Mix, 1 μl of the upstream primer, 1 μl of the downstream primer, and 50 ng of the bacterial genomic DNA.

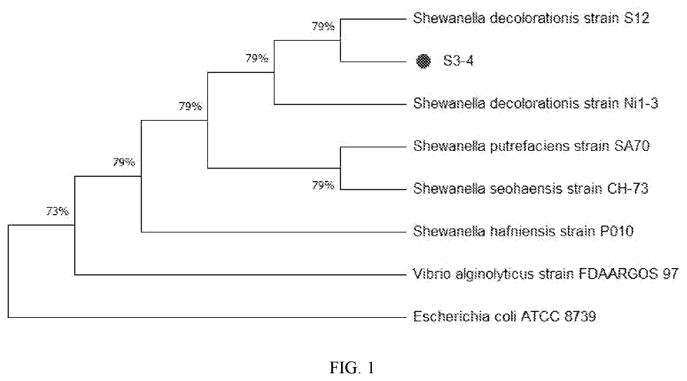

The PCR reaction program was run at 95° C. for 3 minutes; 95° ° C. for 15 seconds, 55° C. for 15 seconds and 72ºC for 30 seconds, totally 35 cycles; and 72ºC for 5 minutes. The amplified product of 16S rDNA full-length sequence was purified and recovered, and the recovered product was cloned in a Tl vector. Escherichia coli was transformed; positive clones were selected for sequencing, sequencing results were compared by BLAST N in GenBank, and the sequences with high homology were obtained. A MEGA X software was utilized for cluster analysis to determine that the isolated strain was Shewanella decolorationis S3-4, and the results were shown in FIG. 1. The isolated strain was Shewanella decolorationis S3-4.

16sDNA sequence of Shewanella decolorationis S3-4 were as follows.

ggcgcgcggctacacatgcagtcgagcggcagcacaagtgagtttactc
atgaggtggcgagcggcggacgggtgagtaatgcctagggatctgccca
gtcgagggggataacagttggaaacgactgctaataccgcatacgccct
acgggggaaaggaggggaccttttggccttccgcgattggatgaaccta
ggtgggattagctagttggtgaggtaatggctcaccaaggcgacgatcc
ctagctgttctgagaggatgatcagccacactgggactgagacacggcc
cagactcctacgggaggcagcagtggggaatattgcacaatgggggaaa
ccctgatgcagccatgccgcgtgtgtgaagaaggccttcgggttgtaaa
gcactttcagtagggaggaaaggttgtaagttaataccttgcagctgtg
acgttacctacagaagaaggaccggctaactccgtgccagcagccgcgg
taatacggagggtccaagcgttaatcggaattactgggcgtaaagcgtg
cgcaggcggtttgttaagcgagatgtgaaagccccgggctcaacctggg
aattgcatttcgaactggcaaactagagtcttgtagaggggggtagaat
tccaggtgtagcggtgaaatgcgtagagatctggaggaataccggtggc
gaaggcggccccctggacaaagactgacgctcaggcacgaaagcgtggg
gagcaaacaggattagataccctggtagtccacgccgtaaacgatgtct
actcggagtttggtgtcttgaacactgggctctcaagctaacgcattaa
gtagaccgcctggggagtacggccgcaaggttaaaactcaaatgaattg
acgggggcccgcacaagcggtggagcatgtggtttaattcgatgcaacg
cgaagaaccttacctactcttgacatccagagaactttccagagatgga
ttggtgccttcgggaactctgagacaggtgctgcatggctgtcgtcagc
tcgtgttgtgaaatgttgggttaagtcccgcaacgagcgcaacccctat
ccttatttgccagcgcgtaatggcgggaactctagggagactgccggtg
ataaaccggaggaaggtggggacgacgtcaagtcatcatggcccttacg
agtagggctacacacgtgctacaatggcgagtacagagggttgcaaagc
cgcgaggtggagctaatctcacaaagctcgtcgtagtccggattggagt
ctgcaactcgactccatgaagtcggaatcgctagtaatcgtgaatcaga
atgtcacggtgaatacgttcccgggccttgtacacaccgcccgtcacac
catgggagtgggctgcaaaagaagtgggtagcttaacctcgggagggcg
c.

A Shewanella decolorationis producing tetrodotoxin, a strain of S3-4, was deposited in the China General Microbiological Culture Collection Center (CGMCC). The deposit address was No. 3, No. 1 Courtyard, Beichen West Road, Chaoyang District, Beijing. The preservation date was Mar. 28, 2022. The deposit number was CGMCC No. 24602.

### Example 2

A fermentation culture method for Shewanella decolorationis S3-4 isolated in Example 1 included the following steps.
(1) Bacteria were inoculated into an LB liquid culture medium.
(2) The LB liquid culture medium was cultured at 28° C. at 200 rpm for 2-3 days.

The LB liquid culture medium included the following components: 10.0 g/L of tryptone, 5.0 g/L of yeast extract powder and 10.0 g/L of sodium chloride.

### Example 3

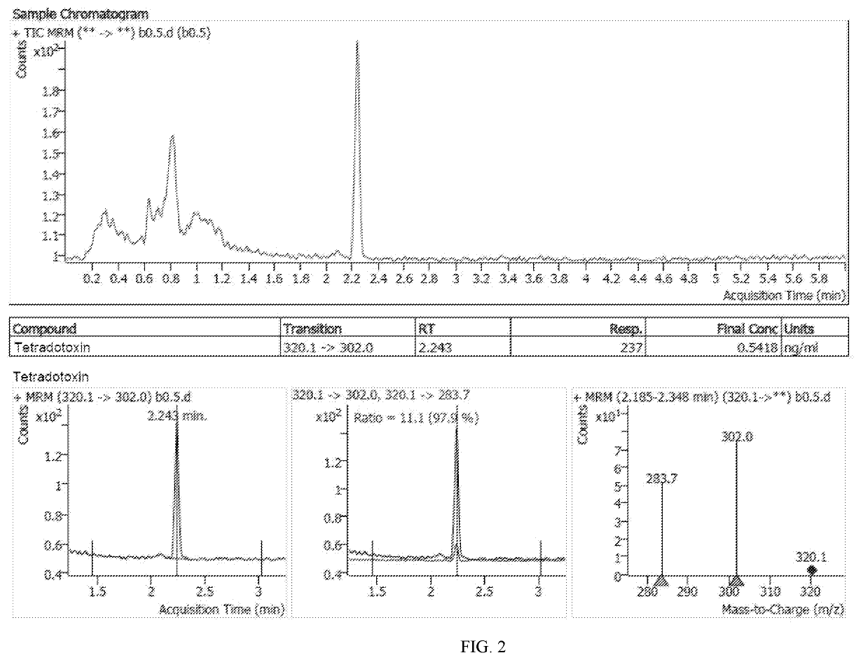

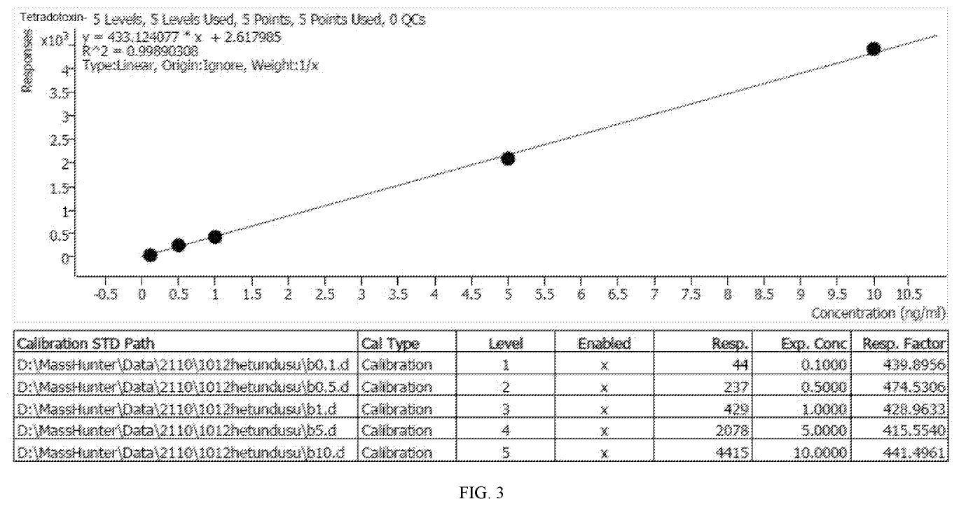

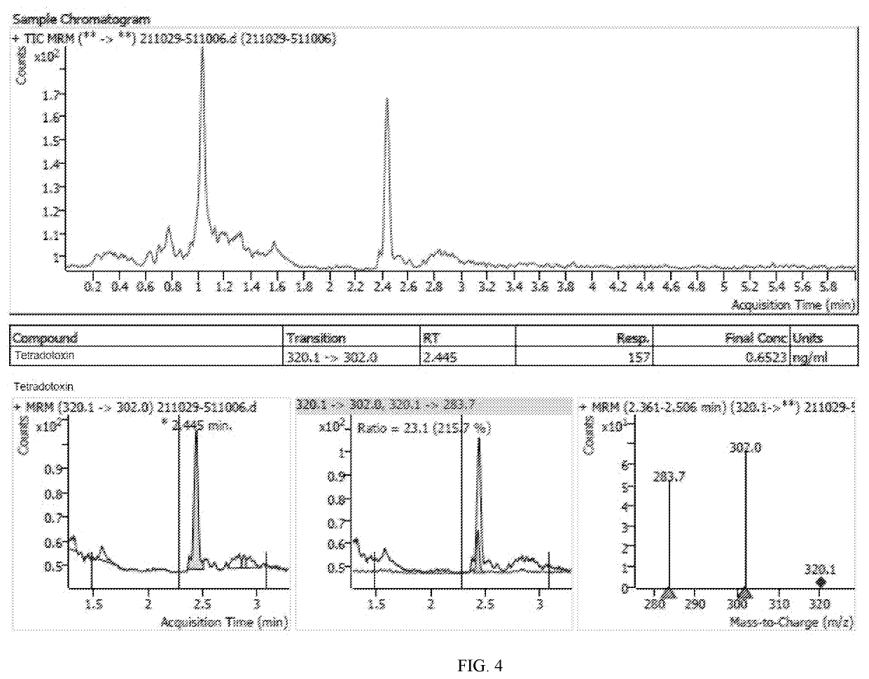

A preparation for tetrodotoxin by utilizing the fermentation of Shewanella decolorationis S3-4 obtained in Example 2 included the following specific steps.
(1) Shewanella decolorationis S3-4 was fermented and cultured, and then centrifuged at 4ºC at 4000×g for 30 minutes to collect strain cells.
(2) 0.1% methanol formate was added and cells were ultrasonically crushed.
(3) The cells were centrifuged at 8000 rpm for 5 minutes.
(4) Supernatant was taken and filtered through an organic filter membrane with a pore size of 0.22 μm.
(5) Tetrodotoxin was detected by liquid chromatography-mass spectrometry.
(6) Liquid chromatography-mass spectrums of a tetrodotoxin standard substance were shown in FIG. 2.
(7) A result of a standard curve was shown in FIG. 3.
(8) Results of liquid chromatography-mass spectrometry for the identification of Shewanella decolorationis-derived tetrodotoxin were shown in FIG. 4, the liquid chromatography-mass spectrometry of the Shewanella decolorationis-derived tetrodotoxin being consistent with that of the standard substance.

In an example of the present disclosure, bacteria were isolated from the ovary, liver and intestinal tissues of Takifugu ocellatus, and screened using the 2216E solid culture medium, TCBS solid culture medium and LB solid culture medium. A Shewanella decolorationis S3-4 capable of producing tetrodotoxin was isolated from the Tetraodontidae body for the first time, and the isolated bacteria were identified by 16S rDNA method.

The tetrodotoxin produced by the isolated Shewanella decolorationis S3-4 was detected by a liquid chromatography-mass spectrometry (LC-MS) method. The detection on a compound of tetrodotoxin by an LC-MS instrument is more accurate. The tetrodotoxin produced by Shewanella decolorationis S3-4 was determined to be a same substance as the tetrodotoxin extracted from Tetraodontidae.

According to the present disclosure, the isolated Shewanella decolorationis S3-4 can be cultured to isolate tetrodotoxin from bacteria on a large scale.

The basic principle, main features and advantages of the present disclosure have been shown and described above. It is to be understood by those skilled in the art that the present disclosure is not limited by the above examples, and what is described in the above examples and specifications only illustrates the principles of the present disclosure, and there will be various changes and improvements in the present disclosure without departing from the spirit and scope of the present disclosure, which fall within the scope of the claimed disclosure. The scope of protection required by the present disclosure is defined by the appended claims and equivalents thereof.

1. A Shewanella decolorationis producing tetrodotoxin, a strain of S3-4, being classified as Shewanella decolorationis and deposited in the China General Microbiological Culture Collection Center (CGMCC) on Mar. 28, 2022 with a deposit number of CGMCC No. 24602.

2. A method for preparing tetrodotoxin by utilizing the Shewanella decolorationis according to claim 1, comprising, inoculating Shewanella decolorationis S3-4 into an LB liquid culture medium, and culturing a same at 28° ° C. at 200 rpm for 2-3 days.

3. The method according to claim 2, wherein the LB liquid culture medium comprises the following components: tryptone with a final concentration of 10.0 g/L, yeast extract powder with a final concentration of 5.0 g/L, and sodium chloride with a final concentration of 10.0 g/L; and the strain of Shewanella decolorationis S3-4 obtained by fermentation culture in the LB liquid culture medium is crushed, isolated and purified to obtain tetrodotoxin.
